(19) United States
(12) Patent Application Publication
(10) Pub. No.: US 2008/0110453 A1
(43) Pub. Date: May 15, 2008

(54) NEBULIZER AND METHODS FOR CONTROLLING THE NEBULIZER

(75) Inventors: David A. Ross, Columbiaville, MI (US); Paul T. Kotnik, Commerce Township, MI (US)

Correspondence Address: CANTOR COLBURN, LLP, 20 Church Street, 22nd Floor, Hartford, CT, 06103, US

(73) Assignee: DELPHI TECHNOLOGIES INC., Troy, MI (US)

(21) Appl. No.: 11/560,150

(22) Filed: Nov. 15, 2006

(51) Int. Cl. A61M 15/00

(52) U.S. Cl. 128/200.16

(57) ABSTRACT

A nebulizer and methods for controlling the nebulizer are provided. In one exemplary embodiment, the nebulizer activates a piezo-electric device to atomize liquid only when a person is inhaling.

### TECHNICAL FIELD

The present invention relates to a nebulizer and methods for controlling the nebulizer.

### BACKGROUND

Nebulizer have been utilized to atomize a liquid. However, when utilized in a medical environment to atomize a medicinal liquid for inhalation by a person, the nebulizer continuously emits atomized liquid which can result in a substantial amount of the atomized liquid not being inhaled by a person.

Accordingly, the inventors herein have recognized a need for an improved nebulizer that minimizes and/or eliminates the above-mentioned deficiency.

### SUMMARY OF THE INVENTION

A nebulizer in accordance with an exemplary embodiment is provided. The nebulizer includes a housing having a reservoir and a chamber. The reservoir is configured to hold a liquid therein. The chamber is in fluid communication with the reservoir and receiving the fluid from the reservoir. The nebulizer further includes a piezo-electric device configured to generate liquid pressure wave pulses in the chamber when the piezo-electric device is activated. The nebulizer further includes a meshed screen disposed proximate the chamber. The nebulizer further includes a sensor configured to generate a first signal indicating whether a person is inhaling proximate the housing. The nebulizer further includes a microprocessor operably associated with the sensor and the piezo-electric device. The microprocessor is configured to activate the piezo-electric device when the first signal indicates the person is inhaling, such that the liquid pressure wave pulses contact the meshed screen and the liquid is atomized as the liquid propagates through the meshed screen.

A method for controlling a nebulizer in accordance with another exemplary embodiment is provided. The nebulizer has a housing with a chamber containing a liquid therein. The nebulizer further includes a piezo-electric device configured to generate liquid pressure wave pulses in the chamber when the piezo-electric device is activated. The nebulizer further includes a meshed screen disposed proximate the chamber. The nebulizer further includes a sensor. The nebulizer further includes a microprocessor operably associated with the sensor and the piezo-electric device. The method includes generating a first signal indicating whether a person is inhaling utilizing the sensor. The method further includes receiving the first signal at the microprocessor. The method further includes activating the piezo-electric device to generate liquid pressure wave pulses in the chamber when the first signal indicates the person is inhaling, utilizing the microprocessor, such that the liquid pressure wave pulses contact the meshed screen and the liquid is atomized as the liquid propagates through the meshed screen.

### BRIEF DESCRIPTION OF THE DRAWINGS

FIG. 1 is a perspective cross-sectional view of a nebulizer in accordance with an exemplary embodiment;

FIG. 2 is an enlarged perspective view of a portion of the nebulizer of FIG. 1;

FIG. 3 is another enlarged perspective view of a portion of the nebulizer of FIG. 1;

FIG. 4 is a perspective view of a nozzle portion utilized in the nebulizer of FIG. 1;

FIG. 5 is an electrical schematic associated with the nebulizer of FIG. 1;

FIG. 6 is a flowchart of a method for controlling the nebulizer in accordance with another exemplary embodiment; and

FIG. 7 is a flowchart of a method for controlling the nebulizer in accordance with another exemplary embodiment.

### DETAILED DESCRIPTION OF EXEMPLARY EMBODIMENTS

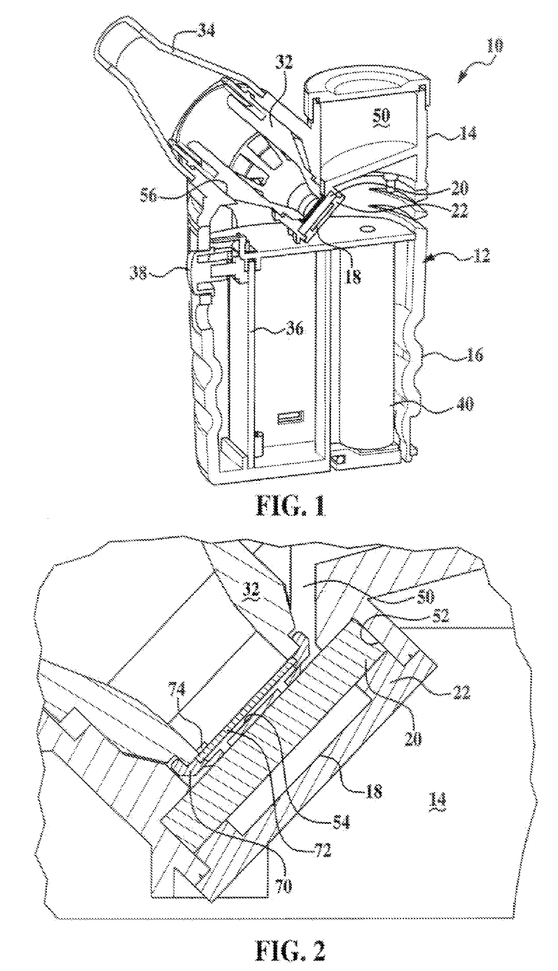

Referring to FIG. 1, perspective cross-sectional view of a nebulizer 10 is provided. The nebulizer 10 includes a housing 12, a piezo-electric device 18, coupling plates 20, 22, a nozzle portion 32, the tube portion 34, a microprocessor 36, a switch 38, and a battery 40.

Referring to FIGS. 1-3, the housing 12 is provided to enclose the remaining components of the nebulizer 10. The housing 12 includes a top housing portion 14 and a bottom housing portion 16.

The top housing portion 14 is coupled to the bottom housing portion 16 utilizing coupling devices such as bolts for example. Of course, in alternative embodiments other fastening means such as weld joints or glue could be utilized to couple the top housing portion 14 to the bottom housing portion 16. The top housing portion 14 is constructed from an injection molded plastic. Of course, in an alternative embodiment, the top housing portion 14 could be constructed from other materials such as stainless steel for example. The top housing portion 14 has a reservoir 50 for holding a liquid therein. Further, the top housing portion 14 has a receiving region 56 for receiving the nozzle portion 32 therein. Further, the top housing a portion 14 has a receiving region 52 communicating with both the reservoir 50 and the receiving region 56. The receiving region 52 is configured to receive the coupling plates 20, 22 and the piezo-electric device 18 therein. A chamber 54 is defined between the coupling plate 20 and the nozzle portion 32, that is in fluid communication with the reservoir 50. The chamber 54 receives liquid from the reservoir 50.

The bottom housing portion 16 is provided to enclose the microprocessor 36, the switch 38 and the battery 40 therein. The bottom housing portion 16 is constructed from an injection molded plastic. Of course, in an alternative embodiment, the bottom housing portion 16 could be constructed from other materials such as stainless steel for example.

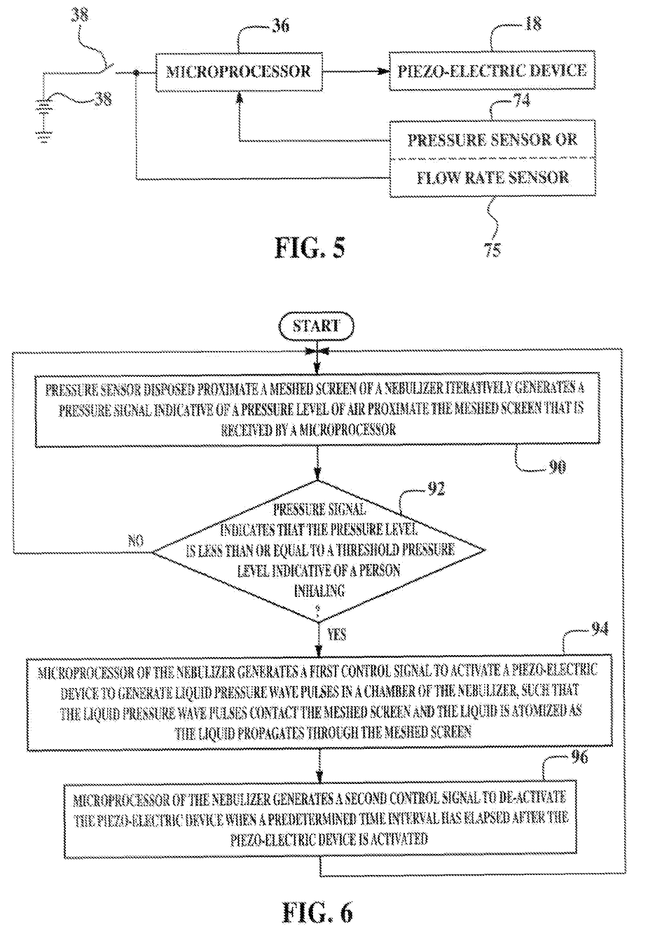

Referring to FIGS. 2 and 5, the piezo-electric device 18 is provided to vibrate in response to a control signal from the microprocessor 36 to generate liquid pressure wave pulses in the chamber 54. The piezo-electric device 18 is electrically coupled to the microprocessor 36 and is physically disposed between the coupling plates 20, 22. The coupling plates 20, 22 are constructed from an injection molded plastic. During operation, the piezo-electric device 18 generates vibrational pulses that the transfer energy through the coupling plate 20 into the liquid in the chamber 54 contacting the coupling plate 20.

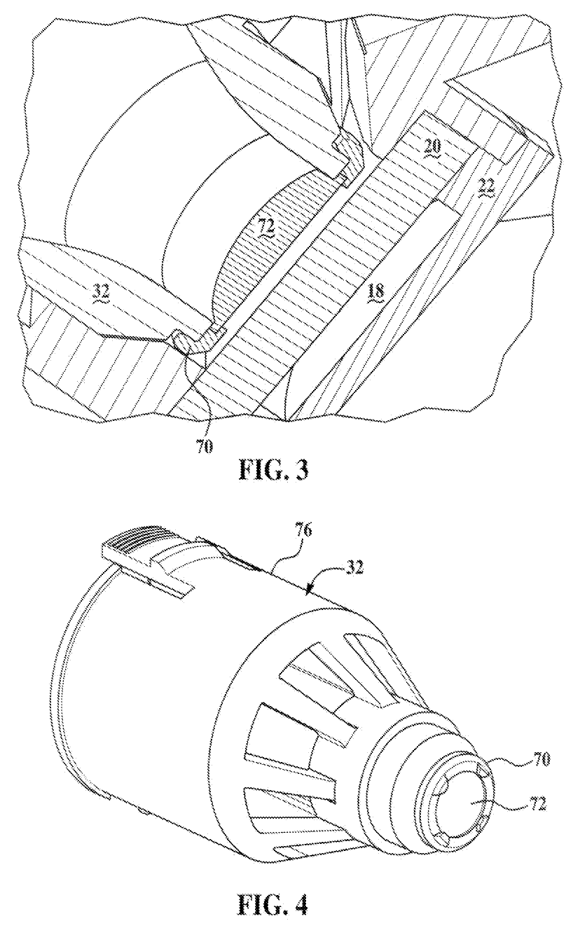

Referring to FIGS. 1, 2 and 4, the nozzle portion 32 is provided to communicate atomized liquid from the top housing portion 14. The nozzle portion 32 includes an offset end portion 70, a meshed screen 72, a pressure sensor 74, and a body portion 76.

the body portion 76 is generally tubular shaped. The offset end portion 70 is disposed on a first end of the body portion 76. The offset end portion 70 is configured to be disposed in the receiving region 56 on the coupling plate 20. The offset end portion 70 includes an aperture extending therethrough. Further, the meshed screen 72 is disposed in the aperture of the offset end portion 70. In one exemplary embodiment, the meshed screen 72 is a substantially flat member having a thickness of approximately 25-100 microns and a plurality of apertures each having a size of approximately 1-3 microns. Of course, other thickness and aperture sizes can be utilized to meet particular operational characteristics. During operation, when liquid pressure wave pulses contact the meshed screen 72, the liquid is atomized as the liquid propagates through the meshed screen 72.

Referring to FIGS. 2 and 5, the pressure sensor 74 is provided to generate a pressure signal indicative of a pressure level of air proximate the meshed screen 72 to detect when an operator of the nebulizer 10 is inhaling. The pressure sensor 74 is coupled to the body portion 76 proximate the meshed screen 72. Further, the pressure sensor 74 is electrically coupled to the microprocessor 36. When the pressure sensor 74 generates a pressure signal indicating that the pressure level is less than or equal to a threshold pressure level, which further indicates that an operator is inhaling, the microprocessor 36 generates a first control signal to activate the piezo-electric device 18. Further, when a predetermined time interval has elapsed after the piezo-electric device 18 is activated, the microprocessor 36 generates a second control signal to de-activate the piezo-electric device 18. In an alternative embodiment, when the pressure sensor 74 generates a pressure signal indicating that the pressure level is greater than the threshold pressure level, which indicates that an operator is not inhaling, the microprocessor generates a second control signal to de-activate the piezo-electric device 18. In an alternative embodiment, the pressure sensor 74 can be disclosed a distance away from the meshed screen 72, and a tube (not shown) can extend from the pressure sensor 74 to a location proximate the meshed screen 72.

Referring to FIG. 1, the tube portion 34 is provided to direct atomized liquid from the nozzle portion 32 outwardly from the nebulizer 10 for inhalation by an operator. The tube portion 34 is configured to be coupled to a second end of nozzle portion 32. The tube portion 34 is constructed from an injection molded plastic. Of course, in an alternative embodiment, the tube portion 34 could be constructed from other materials such as stainless steel for example.

Referring to FIGS. 1 and 5, the battery 40 is electrically coupled through the switch 38 to the microprocessor 36 and the pressure sensor 74. The microprocessor 36 further electrically coupled to the piezo-electric device 18. When the switch 38 has a closed operational position, a voltage from the battery is received by the microprocessor 36 and the pressure sensor 74. Alternately, when the switch 38 has an open operational position, the voltage from the battery is not received by the microprocessor 36 and the pressure sensor 74.

Referring to FIG. 6, a method for controlling the nebulizer 10 utilizing the pressure sensor 74 in accordance with another exemplary embodiment will now be described.

At step 90, the pressure sensor 74 disposed proximate the meshed screen 72 of the nebulizer 10 iteratively generates a pressure signal indicative of a pressure level of air proximate the meshed screen 72 that is received by the microprocessor 36.

At step 92, the microprocessor 36 receives the pressure signal and makes a determination as to whether the pressure signal indicates that the pressure level is less than or equal to a threshold pressure level, indicative of a person inhaling If the value of step 92 equals “yes”, the method of advances to step 94. Otherwise, the method returns to step 90.

At step 94, the microprocessor 36 of the nebulizer 10 generates a first control signal to activate the piezo-electric device 18 to generate liquid pressure wave pulses in the chamber 54 of the nebulizer 10, such that the liquid pressure wave pulses contact the meshed screen and the liquid is atomized as the liquid propagates through the meshed screen 72.

At step 96, the microprocessor 36 of the nebulizer 10 generates a second control signal to de-activate the piezo-electric device 18 when a predetermined time interval has elapsed after the piezo-electric device 18 is activated. After step 96, the method returns to step 90. In an alternative embodiment, the step 96 can be replaced by another step wherein the microprocessor 36 generates a second control signal to de-activate the piezo-electric device 18 when the pressure signal indicates a pressure level greater than the threshold pressure level.

Referring to FIGS. 2 and 5, in an alternative exemplary embodiment, the pressure sensor 74 can be replaced by a flow rate sensor 75. The glow rate sensor 75 is provided to generate a flow rate signal indicative of a flow rate of air proximate the meshed screen 72 to detect when an operator of the nebulizer 10 is inhaling. The flow rate sensor 75 is coupled to the body portion 76 proximate the meshed screen 72. Further, the flow rate sensor 75 is electrically coupled to the microprocessor 36. When the flow rate sensor 75 generates a flow rate signal indicating that the flow rate is greater than a threshold flow rate, which further indicates that an operator is inhaling, the microprocessor 36 generates a first control signal to activate the piezo-electric device 18. Further, when a predetermined time interval has a lapsed after the piezo-electric device 18 is activated, the microprocessor 36 generates a second control signal to de-activate the piezo-electric device 18. In an alternative embodiment, when the flow rate sensor 75 generates a flow rate signal indicating that the flow rate is less than the threshold flow rate, which indicates that an operator is not inhaling, the microprocessor 36 generates a second control signal to de-activate the piezo-electric device 18. In an alternative embodiment, the flow rate sensor 75 can be disclosed a distance away from the meshed screen 72, and a tube (not shown) can extend from the flow rate sensor 75 to a location proximate the meshed screen 72.

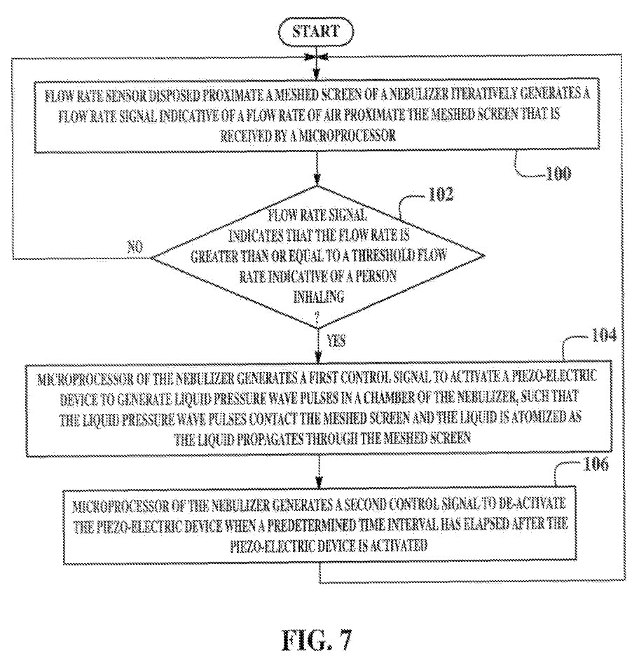

Referring to FIG. 7, a method for controlling the nebulizer 10 utilizing the flow rate sensor 75 in accordance with another exemplary embodiment will now be described.

At step 100, the flow rate sensor 75 disposed proximate the meshed screen 72 of the nebulizer 10 iteratively generates a flow rate signal indicative of a flow rate of air proximate the meshed screen 72 that is received by the microprocessor 36.

At step 102, the microprocessor 36 receives the flow rate signal and makes a determination as to whether the flow rate signal indicates that the flow rate is greater than or equal to a threshold flow rate, indicative of a person inhaling. If the value of step 102 equals “yes”, the method advances to step 104. Otherwise, the method returns to step 100.

At step 104, the microprocessor 36 of the nebulizer 10 generates a first control signal to activate the piezo-electric device 18 to generate liquid pressure wave pulses in the chamber 54 of the nebulizer 10, such that the liquid pressure wave pulses contact the meshed screen 72 and the liquid is atomized as the liquid propagates through the meshed screen 72.

At step 106, the microprocessor 36 of the nebulizer 10 generates a second control signal to de-activate the piezo-electric device 36 when a predetermined time interval has elapsed after the piezo-electric device 36 is activated. After step 106, the method returns to step 100.

The nebulizer and the methods of controlling the nebulizer provide a substantial advantage over other nebulizers and methods. In one exemplary embodiment, the nebulizer 10 provides a technical affect of activating a piezo-electric device to atomize liquid only when a pressure level is less than or equal to a threshold pressure level, indicating that an operator is inhaling. Thus, a substantial portion of the atomize liquid is inhaled by a person, instead of being expelled into the environment and unused by the person.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed herein, but that the invention will include all embodiments falling within the scope of the present application.

What is claimed is:

1. A nebulizer, comprising:
a housing having a reservoir and a chamber, the reservoir configured to hold a liquid therein, the chamber being in fluid communication with the reservoir and receiving the fluid from the reservoir;
a piezo-electric device configured to generate liquid pressure wave pulses in the chamber when the piezo-electric device is activated;
a meshed screen disposed proximate the chamber;
a sensor configured to generate a first signal indicating whether a person is inhaling proximate the housing; and
a microprocessor operably associated with the sensor and the piezo-electric device, the microprocessor configured to activate the piezo-electric device when the first signal indicates the person is inhaling, such that the liquid pressure wave pulses contact the meshed screen and the liquid is atomized as the liquid propagates through the meshed screen.

2. The nebulizer of claim 1, wherein the sensor is a pressure sensor and the first signal is indicative of a pressure level, the first signal indicating the person is inhaling when the pressure level is less than or equal to a threshold pressure level.

3. The nebulizer of claim 2, wherein the microprocessor is further configured to de-activate the piezo-electric device when either the first signal indicates the pressure level is greater than the threshold pressure level or a predetermined time interval has elapsed after the piezo-electric device is activated.

4. The nebulizer of claim 2, wherein the pressure sensor is disposed proximate the meshed screen.

5. The nebulizer of claim 2, further comprising a tube having first and second ends, the first end of the tube being disposed proximate the meshed screen, the second end of the tube being operably coupled to the pressure sensor.

6. The nebulizer of claim 1, wherein the sensor is flow rate sensor and the first signal is indicative of a flow rate, the first signal indicating the person is inhaling when the flow rate is greater than or equal to a threshold flow rate.

7. The nebulizer of claim 6, wherein the microprocessor is further configured to de-activate the piezo-electric device when either the first signal indicates the flow rate is less than the threshold flow rate or a predetermined time interval has elapsed after the piezo-electric device is activated.

8. The nebulizer of claim 6, wherein the flow rate sensor is disposed proximate the meshed screen.

9. The nebulizer of claim 6, further comprising a tube having first and second ends, the first end of the tube being disposed proximate the meshed screen, the second end of the tube being operably coupled to the flow rate sensor.

10. The nebulizer of claim 1, wherein the microprocessor generates a control signal to induce the piezo-electric device to be activated.

11. A method for controlling a nebulizer, the nebulizer having a housing with a chamber containing a liquid therein, the nebulizer further having a piezo-electric device configured to generate liquid pressure wave pulses in the chamber when the piezo-electric device is activated, the nebulizer further having a sensor, the nebulizer further having a microprocessor operably associated with the sensor and the piezo-electric device, the method comprising:
generating a first signal indicating whether a person is inhaling utilizing the sensor;
receiving the first signal at the microprocessor; and
activating the piezo-electric device to generate liquid pressure wave pulses in the chamber when the first signal indicates the person is inhaling, utilizing the microprocessor, such that the liquid pressure wave pulses contact the meshed screen and the liquid is atomized as the liquid propagates through the meshed screen.

12. The method of claim 11, wherein the sensor is a pressure sensor and the first signal is indicative of a pressure level, the first signal indicating the person is inhaling when the pressure level is less than the equal to a threshold pressure level.

13. The method of claim 12, further comprising de-activating the piezo-electric device when either the first signal indicates the pressure level is greater than the threshold pressure level or a predetermined time interval has elapsed after the piezo-electric device is activated, utilizing the microprocessor.

14. The method of claim 11, wherein the sensor is a flow rate sensor and the first signal is indicative of a flow rate, the first signal indicating the person is inhaling when the flow rate is greater than or equal to a threshold flow rate.

15. The method of claim 14, further comprising de-activating the piezo-electric device when either the first signal indicates the flow rate is less than the threshold flow rate or a predetermined time interval has elapsed after the piezo-electric device is activated, utilizing the microprocessor.
